(19) United States
(12) Patent Application Publication
(10) Pub. No.: US 2008/0317865 A1
(43) Pub. Date: Dec. 25, 2008

(54) Quench liquids and washing systems for production of microparticles

(75) Inventors: Kristen Trapani, Arlington, MA (US); Paul Herbert, Wayland, MA (US)

Correspondence Address: ELMORE PATENT LAW GROUP, PC, 515 Groton Road, Unit 1R, Westford, MA, 01886, US

(73) Assignee: ALKERMES, INC., Cambridge, MA (US)

(21) Appl. No.: 12/140,542

(22) Filed: Jun. 17, 2008

Related U.S. Application Data
(60) Provisional application No. 60/945,188, filed on Jun. 20, 2007.

(51) Int. Cl. A61K 9/14; A61K 38/02; A61K 38/16; A61K 39/395; A61K 38/20; A61K 38/21; A61K 38/14; A61K 38/28; A61K 38/43; A61K 38/08; A61P 43/00

(52) U.S. Cl. 424/499; 424/501; 514/2; 514/12; 424/130.1; 424/85.2; 424/85.4; 514/8; 514/3; 424/94.1; 514/17

(57) ABSTRACT

The present invention provides coacervation methods forming compositions for the sustained release water soluble active agents, including biologically active polypeptides. The invention further relates to the discovery of improved non-aqueous quench liquids and washing systems, which enable a reduction in the amount and concentration of hardening agents such as heptane used to produce microparticles, while providing acceptable product yields and residual solvent levels.

### RELATED APPLICATION

This application claims the benefit of U.S. Provisional Application No. 60/945,188, filed on Jun. 20, 2007. The entire teaching of the above application is incorporated herein by reference.

### BACKGROUND OF THE INVENTION

Numerous proteins and peptides, collectively referred to herein as polypeptides, exhibit biological activity in vivo and are useful as medicaments. Many illnesses or conditions require maintenance of a sustained level of medicament to provide the most effective prophylactic and/or therapeutic effects. Sustained levels are often achieved by the administration of biologically active polypeptides by frequent subcutaneous injections, which often results in fluctuating levels of medicament and poor patient compliance.

As an alternative, the use of biodegradable materials, such as polymers, encapsulating the medicament can be employed as a sustained delivery system. The use of biodegradable polymers, for example, in the form of microparticles or microcarriers, can provide a sustained release of medicament, by utilizing the inherent biodegradability of the polymer to control the release of the medicament thereby providing a more consistent, sustained level of medicament and improved patient compliance.

A variety of methods is known by which compounds can be encapsulated in the form of microparticles. In these methods, the material to be encapsulated (drugs or other active agents) is generally dissolved, dispersed, or emulsified, using stirrers, agitators, or other dynamic mixing techniques, in a solvent containing the wall forming material. Solvent is then removed from the microparticles and thereafter the microparticle product is obtained.

Many of the published procedures for microencapsulation with biodegradable polymers employ solvent evaporation/extraction techniques, wherein an oil phase comprising the active agent and the polymer in an organic solvent is dispersed in a continuous aqueous phase. The solvent diffuses out of the oil phase droplets, resulting in the formation of microparticles. These techniques are particularly suitable for water insoluble drugs because these drugs will tend not to partition into the continuous aqueous phase. On the other hand, water soluble drugs may partially partition into the aqueous phase during the preparation process, resulting in a low encapsulation efficiency.

Non-solvent induced coacervation, or phase separation, referred to herein as coacervation, is a method which has frequently been employed to prepare microparticles comprised of a biodegradable polymeric matrix and a water soluble biologically active agent. The coacervation method utilizes a continuous phase that is a non-solvent for the polymer and in which hydrophilic active agents also are not soluble. Drug partitioning into the continuous phase does not occur to an appreciable extent, and relatively high encapsulation efficiencies are typical.

In a conventional coacervation process, a known amount of polymer, such as poly-(lactide-co-glycolide), PLG, with a monomeric ratio of lactide to glycolide ranging from 100:0 to 50:50, is dissolved in an appropriate organic solvent. A solid drug, preferably lyophilized and micronized, may be dispersed in the polymer solution, where it is insoluble or slightly soluble in the organic solvent. Alternatively, the active agent may be dissolved in water, or in water which contains some additives, and emulsified in the polymer solution, forming a water-in-oil emulsion. This emulsion may be referred to as the inner emulsion. The resultant suspension or inner emulsion, also referred to herein as a first phase, is then added to a reactor and addition of a non-solvent, or coacervation agent, is initiated at a predetermined rate. Addition of the coacervation agent results in the formation of a dispersion of coacervate droplets containing polymer, active agent and polymer solvent. The coacervate droplets are also referred to as nascent microparticles or embryonic microparticles. At the completion of the coacervation agent addition, the mixture, referred to herein as a coacervate, is transferred into a quench liquid containing a hardening agent to solidify the semi-solid microspheres. The hardened microspheres are collected, washed, and dried to remove solvents to acceptable levels.

The coacervation process generally provides good encapsulation efficiency for water-soluble active agents and can be optimized to produce microparticles that are acceptable with respect to critical attributes including particle size distribution, and the time course of drug release in vitro or after injection into a patient. However, the removal of residual solvents, including halogenated solvents commonly used to dissolve the polymer, from microparticles formed by coacervation can be inefficient. Efforts to lower residual solvent levels can result in lower microparticle yields due to additional processing, or lower potency due to partial extraction of the active agent during solvent removal.

In addition, coacervation processes known in the art consume large volumes of organic solvents when carried out at a commercial scale. A major contributor to solvent consumption is the quench step, which typically employs heptane as a hardening solvent. Heptane is sometimes subject to worldwide shortages. Moreover, the quantity of microparticles that can be produced in a single batch is often limited by the capacity of the quench tank. Furthermore, pure heptane does not dissipate static charge well, which may present a fire hazard.

Thus, a need exists for a coacervation process that produces high yields of microparticles with acceptable residual solvent levels and minimal losses of active agent during processing. Moreover, there is a need for coacervation processes that are efficient with respect to consumption of process solvents and tank capacity.

Methods for reducing residual solvent levels in microparticle formulations made by coacervation or other methods are known in the art. For example, U.S. Pat. Nos. 5,792,477 and 5,916,598 disclose a process whereby microparticles are contacted with an aqueous washing system to reduce the level of residual organic solvent to less than about 2% by weight of the microparticles. The aqueous washing system is water, or an aqueous solution of water and a solvent for the residual solvent in the microparticles. The aqueous washing system is at a temperature in the range of from about 25° C. to about 40° C. The organic solvent used in such a process is preferably a non-halogenated solvent, and most preferably benzyl alcohol alone or in combination with ethyl acetate.

Because the processes disclosed in U.S. Pat. Nos. 5,792,477 and 5,916,598 use an aqueous washing system to reduce solvent levels, it suffers from the drawback that it may result in unacceptable depletion of water soluble active agents, such as peptides, from the microparticles.

U.S. Pat. No. 6,824,822 discloses non-aqueous washing systems capable of reducing residual solvent levels from microparticles containing a water-soluble active agent while maintaining acceptable levels of the active agents. Processes are disclosed wherein microparticles are formed by extracting a halogenated solvent from a coacervate with an extraction medium, the extraction medium being a non-solvent for the polymer and a solvent for the halogenated solvent and the coacervating agent. The microparticles are then washed with a non-aqueous washing system that is either 100% ethanol, a non-aqueous solvent blend comprising ethanol, or a blend of ethanol and heptane.

In addition, U.S. Pat. No. 6,824,822 discloses processes wherein a coacervate containing nascent microparticles is transferred to a quench liquid comprising a blend of a hardening solvent, such as heptane, and a washing solvent, such as ethanol, in order to harden and wash the microparticles in a single step. Such processes may include a step of rinsing the microparticles with the hardening solvent after the extracting step. The quench liquids specifically disclosed for hardening and washing in a single step are a blend of 90% heptane and 10% ethanol, and a blend of 95% heptane and 5% ethanol. While U.S. Pat. No. 6,824,822 teaches preferred solvent blends made up of from 50% heptane and 50% ethanol to 95% heptane and 5% ethanol, microspheres quenched with a blend of 50% heptane and 50% ethanol and washed with 100% heptane had unacceptable handling characteristics. No commercially successful processes are disclosed wherein a coacervate is extracted with a quench liquid containing more than 10% ethanol by weight.

US Patent Application No. 20060110423, incorporated herein by reference, discloses compositions for the sustained release of biologically active polypeptides, and methods of forming and using said compositions, for the sustained release of biologically active polypeptides. The sustained release compositions comprise a biocompatible polymer, and agent, such as a biologically active polypeptide, and a sugar. The agent and sugar are dispersed in the biocompatible polymer separately or, preferably, together. In a particular embodiment, the sustained release composition is characterized by a release profile having a ratio of maximum serum concentration (Cₘₐₓ) to average serum concentration (Cₐᵥₑ) of about 3 or less.

The aforementioned US Patent Application discloses a process for forming a composition for the sustained release of biologically active polypeptide. In this process, an aqueous phase comprising water, a water soluble polypeptide and a sugar, is combined with an oil phase comprising a biocompatible polymer and a solvent for the polymer, forming a water-in-oil emulsion. A coacervation agent, for example silicone oil, vegetable oil or mineral oil, is added to the mixture to form embryonic microparticles; which are subsequently transferred to a quench liquid composed of either pure heptane or a mixture of 90% heptane and 10% ethanol (w/w) to undergo hardening. The hardened microparticles are then collected, washed with pure heptane, and dried. The disclosed process is conducted in a batch mode using stirred tank reactors and ranges in scale from 100 gram to 1 kg. While this process can yield microparticles with good to excellent characteristics of particle size, residual solvents and drug release kinetics, it employs large volumes of the 90% heptane, 10% ethanol blend as a quench liquid and 100% heptane for washing the microparticles. As a result, the process consumes large quantities of heptane. Moreover, both liquids dissipate static charge poorly. In addition, the batch size of microparticles is limited by the capacity of the quench tank. The ratio of quench liquid to polymer solvent is typically at least 16:1 by weight in order to afford an acceptable microparticle yield. An improved solvent system for microparticle quenching would potentially enable a lower ratio of quench liquid to polymer solvent, which would in turn enable a larger microparticle batch size for a given quench tank capacity.

The documents described above all disclose coacervation processes that can be used to prepare microparticles that contain an active agent and acceptable levels of residual solvents. These processes either employ aqueous washing systems, which can result in depletion of water-soluble active agents; or single or multiple step non-aqueous quench and wash steps that employ large quantities of 100% heptane or blends of heptane and an alcohol that comprise at least 90% heptane by weight. Accordingly, the need still exists for coacervation processes that produce high yields of microparticles with acceptable handling characteristics and residual solvent levels, and minimal losses of active agent, that are efficient with respect to consumption of process solvents and tank capacity, and that avoid or minimize the use of 100% heptane or solvent blends comprising at least 90% heptane.

### SUMMARY OF THE INVENTION

This invention relates to coacervation processes for forming compositions for the sustained release of water soluble active agents, including biologically active polypeptides. The invention further relates to the discovery of improved non-aqueous quench liquids and washing systems, which enable a reduction in the amount and concentration of hardening agents such as heptane used to produce microparticles, while providing acceptable product yields and residual solvent levels.

One aspect of the invention is a method for preparing microparticles comprising:
(a) providing a first phase comprising an active agent, a biocompatible polymer and a polymer solvent;
(b) forming a coacervate; and
(c) combining the coacervate with a quench liquid comprising a hardening solvent and between about 15% and about 45% by weight of a washing solvent, thereby forming microparticles containing the active agent.

This aspect of the invention includes a method for forming compositions for the sustained release of biologically active agents, such as polypeptides, which comprises forming a first phase comprising the active agent, a polymer and a solvent; adding a coacervation agent to form embryonic microparticles; transferring the embryonic microparticles to a quench liquid comprising a hardening solvent and a washing solvent to harden the microparticles; collecting the hardened microparticles; and drying the microparticles. The first phase can be a water-in-oil emulsion prepared by dispersing, for example, by sonication or homogenization, an aqueous solution of the active agent in an organic solution comprising a biocompatible polymer and a solvent for the polymer. When the first phase is a water-in-oil emulsion, it can also be referred to as the inner emulsion or the primary emulsion. Alternatively, the first phase can be a suspension wherein the drug in the solid state is dispersed in an organic solution comprising a biocompatible polymer and a solvent for the polymer.

In a particular embodiment, the hardening solvent employed in step (c) above is heptane and the washing solvent is ethanol. Additionally or alternatively, the microparticles formed in step (c) above are washed with a hardening solvent, a washing solvent or a blend thereof prior to drying.

Another aspect of the invention is a method for preparing microparticles comprising:
(a) providing a first phase comprising an active agent, a biocompatible polymer and a polymer solvent;
(b) forming a coacervate; and
(c) combining the coacervate with a quench liquid comprising a hardening solvent and a washing solvent, thereby forming microparticles containing the active agent; and
(d) washing the microparticles with a washing solvent or a solvent mixture comprising a washing solvent.

In a particular embodiment, the hardening solvent employed in step (c) above is heptane and the washing solvent is ethanol. Additionally or alternatively, the washing solvent employed in step (d) above is ethanol. Additionally or alternatively, the solvent mixture employed in step (d) above also comprises a hardening solvent. Additionally or alternatively, the quench liquid of step (c) above and the solvent mixture of step (d) above both comprise heptane and ethanol, in either the same or differing proportions.

This invention relates to methods for forming compositions for the sustained release of agents, such as biologically active polypeptides. The sustained release compositions of this invention comprise a biocompatible polymer, and an agent, such as a biologically active polypeptide. In a preferred embodiment, the biologically active polypeptide is an antidiabetic or glucoregulatory polypeptide, such as GLP-1, GLP-2, exendin-3, exendin-4 or an analog, derivative or agonist thereof, preferably exendin-4.

The sustained release composition may additionally comprise one or more excipients, including but not limited to salts, sugars, carbohydrates, buffers and surfactants. The excipient is preferably sucrose, mannitol or a combination thereof. A preferred combination includes exendin-4 and sucrose and/or mannitol.

Additionally or alternatively, the sustained release composition consists essentially of or, alternatively consists of, a biocompatible polymer, exendin-4 at a concentration of about 3% w/w and sucrose at a concentration of about 2% w/w. The biocompatible polymer is preferably a poly-lactide-coglycolide polymer.

The agent or polypeptide, e.g. exendin-4, can be present in the composition described herein at a concentration of about 0.01% to about 10% w/w based on the total weight of the final composition. In addition, the sugar, e.g. sucrose, can be present in a concentration of about 0.01% to about 5% w/w of the final weight of the composition.

The compositions of this invention can be administered to a human, or other animal, by injection, implantation (e.g., subcutaneously, intramuscularly, intraperitoneally, intracranially, and intradermally), administration to mucosal membranes (e.g., intranasally, intravaginally, intrapulmonary or by means of a suppository), or in situ delivery (e.g., by enema or aerosol spray).

When the sustained release composition has incorporated therein a hormone, particularly an anti-diabetic or glucoregulatory peptide, for example, GLP-1, GLP-2, exendin-3, exendin-4 or agonists, analogs or derivatives thereof, the composition is administered in a therapeutically effective amount to treat a patient suffering from diabetes mellitus, impaired glucose tolerance (IGT), obesity, cardiovascular (CV) disorder or any other disorder that can be treated by one of the above polypeptides or derivatives, analogs or agonists thereof.

The use of a sugar in the sustained release compositions of the invention improves the bioavailability of the incorporated biologically active polypeptide, e.g., anti-diabetic or glucoregulatory peptides, and minimizes loss of activity due to instability and/or chemical interactions between the polypeptide and other components contained or used in formulating the sustained release composition, while maintaining an excellent release profile.

The advantages of the sustained release formulations as described herein include increased patient compliance and acceptance by eliminating the need for repetitive administration, increased therapeutic benefit by eliminating fluctuations in active agent concentration in blood levels by providing a desirable release profile, and a potential lowering of the total amount of biologically active polypeptide necessary to provide a therapeutic benefit by reducing these fluctuations.

### BRIEF DESCRIPTION OF THE DRAWINGS

FIG. 1 is a chart displaying microparticle yields at a quench liquid:polymer solvent ratio of 16:1.

FIG. 2 is a chart displaying microparticle yields at a quench liquid:polymer solvent ratio of 12:1.

FIG. 3 is a chart displaying residual solvent levels for microparticles made using a quench liquid:polymer solvent ratio of 16:1.

FIG. 4 is a chart displaying residual solvent levels for microparticles made using a quench liquid:polymer solvent ratio of 12:1.

FIG. 5 is a chart displaying particle size data for microparticles made using a quench liquid:polymer solvent ratio of 16:1.

FIG. 6 is a chart displaying particle size data for microparticles made using a quench liquid:polymer solvent ratio of 12:1.

### DETAILED DESCRIPTION OF THE INVENTION

This invention relates to methods of forming compositions for the sustained release of biologically active agents, including water soluble active agents such as polypeptides. The invention further relates to the discovery of improved non-aqueous quench liquids and washing systems, which enable a reduction in the amount and concentration of hardening agents such as heptane used to produce microparticles, while providing acceptable product yields and residual solvent levels.

### The Agent

Biologically active polypeptides as used herein collectively refers to biologically active proteins and peptides and the pharmaceutically acceptable salts thereof, which are in their molecular, biologically active form when released in vivo, thereby possessing the desired therapeutic, prophylactic and/or diagnostic properties in vivo. Typically, the polypeptide has a molecular weight between 500 and 200,000 Daltons.

Suitable biologically active polypeptides include, but are not limited to, glucagon, glucagon-like peptides such as, GLP-1, GLP-2 or other GLP analogs, derivatives or agonists of glucagons-like peptides, exendins, such as exendin-3 and exendin-4, derivatives, agonists and analogs thereof, vasoactive intestinal peptide (VIP), immunoglobulins, antibodies, cytokines (e.g., lymphokines, monokines, chemokines), interleukins, macrophage activating factors, interferons, erythropoietin, nucleases, tumor necrosis factor, colony stimulating factors (e.g., G-CSF), insulin, enzymes (e.g., superoxide dismutase, plasminogen activator, etc.), tumor suppressors, blood proteins, hormones and hormone analogs and agonists (e.g., follicle stimulating hormone, growth hormone, adrenocorticotropic hormone, and luteinizing hormone releasing hormone (LHRH)), vaccines (e.g., tumoral, bacterial and viral antigens), antigens, blood coagulation factors, growth factors (NGF and EGF), gastrin, GRH, antibacterial peptides such as defensin, enkephalins, bradykinins, calcitonin and muteins, analogs, truncation, deletion and substitution variants and pharmaceutically acceptable salts of all the foregoing.

Alternatively, the polypeptide can be generally selected from coagulation modulators, cytokines, endorphins, kinins, hormones, luteinizing hormone-releasing hormone analogs and others. Coagulation modulators include, for example, α-1-antitrypsin, α-2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, thrombomodulin and combinations thereof. When applicable, both the “active” and “inactive” versions of these proteins are included.

Preferred cytokines include, without limitation, colony stimulating factor 4, heparin binding neurotrophic factor (HBNF), interferons, interleukins, tumor necrosis factor, granuloycte colony-stimulating factor (G-CSF), granulocyte-macrophage colony-stimulating factor (GM-CSF), macrophage colony-stimulating factor, midkine (MD), thymopoietin and combinations thereof.

Preferred endorphins include, but are not limited to, dermorphin, dynorphin, α-endorphin, β-endorphin, γ-endorphin, σ-endorphin, enkephalin, substance P, and combinations thereof.

Preferred peptidyl hormones include activin, amylin, angiotensin, atrial natriuretic peptide (ANP), calcitonin, calcitonin gene-related peptide, calcitonin N-terminal flanking peptide, cholecystokinin (CCK), ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, ghrelin, glucogon, gonadotropin-releasing factor (GnRF or GNRH), growth hormone releasing factor (GRF, GRH), human chorionic gonadotropin (hCH), inhibin A, inhibin B, insulin, leptin, lipotropin (LPH), luteinizing hormone (LH), luteinizing hormone-releasing hormone, melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, triiodothyronine, vasoactive intestinal peptide (VIP), vasopressin (antidiuretic hormone, ADH) and combinations thereof.

Particularly preferred analogues of LHRH include buserelin, deslorelin, fertirelin, goserelin, histrelin, leuprolide (leuprorelin), lutrelin, nafarelin, tryptorelin and combinations thereof. Particularly preferred kinins include bradykinin, potentiator B, bradykinin potentiator C, kallidin and combinations thereof.

Still other peptidyl drugs that provide a desired pharmacological activity can be incorporated into the delivery systems of the invention. Examples include abarelix, adenosine deaminase, anakinra, ancestim, alteplase, alglucerase, asparaginase, bivalirudin, bleomycin, bombesin, desmopressin acetate, des-Q14-ghrelin, dornase-α., enterostatin, erythropoietin, fibroblast growth factor-2, filgrastim, β-glucocerebrosidase, gonadorelin, hyaluronidase, insulinotropin, lepirudin, magainin I, magainin II, nerve growth factor, pentigetide, thrombopoietin, thymosin α-1, thymidin kinase (TK), tissue plasminogen activator, tryptophan hydroxylase, urokinase, urotensin II and combinations thereof.

Exendin-4 is a 39 amino acid polypeptide. The amino acid sequence of exendin-4 can be found in U.S. Pat. No. 5,424,286 issued to Eng on Jun. 13, 1995, the entire content of which is hereby incorporated by reference. Exendin-4 has been shown in humans and animals to stimulate secretion of insulin in the presence of elevated blood glucose concentrations, but not during periods of low blood glucose concentrations (hypoglycemia). It has also been shown to suppress glucagon secretion, slow gastric emptying and affect food intake and body weight, as well as other actions. As such, exendin-4 and analogs and agonists thereof can be useful in the treatment of diabetes mellitus, IGT, obesity, etc.

The amount of biologically active polypeptide, which is contained within the polymeric matrix of a sustained release composition, is a therapeutically, diagnostically or prophylactically effective amount which can be determined by a person of ordinary skill in the art, taking into consideration factors such as body weight, condition to be treated, type of polymer used, and release rate from the polymer.

Sustained release compositions generally contain from about 0.01% (w/w) to about 50% (w/w) of the agent, e.g., biologically active polypeptide (such as exendin-4) (total weight of composition). For example, the amount of biologically active polypeptide (such as exendin-4) can be from about 0.1% (w/w) to about 30% (w/w) of the total weight of the composition. The amount of polypeptide will vary depending upon the desired effect, potency of the agent, the planned release levels, and the time span over which the polypeptide will be released. Preferably, the range of loading is between about 0.1% (w/w) to about 10% (w/w), for example, 0.5% (w/w) to about 5% (w/w). Superior release profiles were obtained when the agent, e.g. exendin-4, was loaded at about 3% w/w.

### The Polymer

Polymers suitable to form the sustained release composition of this invention are biocompatible polymers which can be either biodegradable or non-biodegradable polymers or blends or copolymers thereof. A polymer is biocompatible if the polymer and any degradation products of the polymer are non-toxic to the recipient and also possess no significant deleterious or untoward effects on the recipient's body, such as a substantial immunological reaction at the injection site.

Biodegradable, as defined herein, means the composition will degrade or erode in vivo to form smaller units or chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanhydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl fluoride, poly(vinyl imidazole), chlorosulphonate polyolefins, polyethylene oxide, blends thereof, and copolymers thereof.

Acceptable molecular weights for polymers used in this invention can be determined by a person of ordinary skill in the art taking into consideration factors such as the desired polymer degradation rate, physical properties such as mechanical strength, end group chemistry and rate of dissolution of polymer in solvent. Typically, an acceptable range of molecular weight is of about 2,000 Daltons to about 2,000,000 Daltons. In a preferred embodiment, the polymer is biodegradable polymer or copolymer. In a more preferred embodiment, the polymer is a poly(lactide-co-glycolide) (hereinafter “PLG”) with a lactide:glycolide mole ratio of about 1:1 and a molecular weight of about 10,000 Daltons to about 90,000 Daltons. A PLG copolymer with a 1:1 lactide:glycolide mole ratio can also be referred to as a 50:50 PLG. In an even more preferred embodiment, the PLG used in the present invention has a molecular weight of about 30,000 Daltons to about 70,000 Daltons such as about 50,000 to about 60,000 Daltons.

The PLGs can possess carboxylic acid end groups or blocked end groups, such as can be obtained by means known in the art, including esterifying the acid or employing an alkyl alcohol as initiator in the ring-opening polymerization of DL-lactide and glycolide.

Polymers can also be selected based upon the polymer's inherent viscosity. Suitable inherent viscosities include about 0.06 to 1.0 dL/g, such as about 0.2 to 0.6 dL/g, more preferably between about 0.3 to 0.5 dL/g. Preferred polymers are chosen that will degrade in 3 to 4 weeks. Suitable polymers can be purchased from Lakeshore Biomaterials, Inc. (Birmingham, Ala.), such as those sold as 5050 DL 3A or 5050 DL 4A. Boehringer Ingelheim Resomer PLGs may also be used, such as Resomer™ RG503 and 503H.

It is known in the art (see, for example, Peptide Acylation by Poly(α-Hydroxy Esters) by Lucke et al., Pharmaceutical Research, Vol. 19, No. 2, p. 175-181, February 2002) that proteins and peptides which are incorporated in PLG matrices can be undesirably altered (e.g., degraded or chemically modified) as a result of interaction with degradation products of the PLG or impurities remaining after preparation of the polymer, such as, for example, unreacted lactide or glycolide. As such, the PLG polymers used in the preparation of microparticle formulations may be purified using art recognized purification methods.

The sustained release composition of this invention is a microparticle. A microparticle, as defined herein, comprises a polymer component having a diameter of less than about one millimeter and having biologically active polypeptide dispersed or dissolved therein. A microparticle can have a spherical, non-spherical or irregular shape. Typically, the microparticle will be of a size suitable for injection. A typical size range for microparticles is 1000 microns or less. In a particular embodiment, the microparticle ranges from about one to about 180 microns in diameter.

### Additional Excipients

Additional excipients or additives may be included in the microparticle compositions of the invention to serve a multiplicity of functions, including stabilization of the active agent during the encapsulation process, during storage prior to use, or during the period after injection and prior to release when the active agent resides in the microparticle at body temperature under moist conditions. Moreover, excipients can increase or decrease the rate of release of the agent. Ingredients which can substantially increase the rate of release include pore forming agents and excipients which facilitate polymer degradation. For example, the rate of polymer hydrolysis is increased in non-neutral pH. Therefore, an acidic or a basic excipient such as an inorganic acid or inorganic base can be added to the polymer solution, used to form the microparticles, to alter the polymer erosion rate. Ingredients which can substantially decrease the rate of release include excipients that decrease the water solubility of the agent. Excipients may also be employed to improve the biocompatibility and local tolerability of the microparticle composition.

Suitable excipients include, for example, salts, including buffer salts, sugars, carbohydrates, and surfactants, and are known to those skilled in the art. An acidic or a basic excipient may also be suitable. The amount of excipient used can be based on ratio to the biologically active polypeptide agent, on a weight basis and can be determined by one of skill in the art using available methods. Alternatively, the amount of excipient can be based on its content as a percent of the microparticle dry weight. The combined loading of the active agent and the excipient, or excipients if more than one is present, may have an effect on the release profile of the active agent. For example, when the combined loading of the active agent and the excipients exceeds about 10% of the total dry weight of the microparticles, a greater portion of drug may be released immediately upon suspension of the microparticles in a diluent for injection, or during the first day after injection. In a preferred embodiment, the combined loading of active agent and excipients is about 10% of the total dry weight of the microparticles. In a more preferred embodiment, the combined loading of active agent and excipients is from about 3% to about 8% of the total dry weight of the microparticles. In a still more preferred embodiment, the combined loading of active agent and excipients is from about 5% to about 7% of the total dry weight of the microparticles. Superior release profiles were obtained when an active agent, e.g. exendin-4, was loaded together with sucrose at a combined loading of from about 5% to about 7% w/w.

Excipients can be incorporated into the microparticle compositions of the present invention by several different means. In a preferred embodiment, water-soluble excipients are dissolved together with the active agent in water and then dispersed in the polymer solution prior to the addition of the coacervation agent. Alternatively, excipients can be added as solids at any stage of the process, or dissolved in the polymer solution, or dissolved in water and dispersed in the polymer solution separately from the active agent.

### Salts

Buffer salt, as defined herein is the salt remaining following removal of solvent from a buffer. Buffers are solutions containing either a weak acid and a related salt of the acid, or a weak base and a salt of the base. Buffers can maintain a desired pH to assist in stabilizing the formulation. This maintenance of pH can be afforded during processing, storage and/or release. For example, the buffer can be monobasic phosphate salt or dibasic phosphate salt or combinations thereof or a volatile buffer such as ammonium bicarbonate. Other buffers include, but are not limited to, acetate, citrate, succinate and amino acids such as glycine, arginine and histidine. The buffer when present in the final sustained release composition can range from about 0.01% to about 10% of the total weight.

Salting-out salts can also be employed as excipients in the compositions of the present invention. Salting-out salts, as that term is used herein, refers to salts which are in the Hofmeister series of precipitants of serum as described in Thomas E. Creighton in Proteins: Structures and Molecular Principles, pp. 149-150 (published by W.H. Freeman and Company, New York). In general, the salting-out salts are known in the art as suitable for precipitating a protein, without denaturing the protein. Salting-out salts can also be described in terms of the “kosmotrope” and “chaotrope” properties of the constituent ions. The term kosmotrope generally refers to a solute that stabilizes proteins and chaotrope describes a solute that is destabilizing. Kosmotropic ions have a high charge density (e.g., SO₄²⁻, HPO₄²⁻, Mg²⁺, Ca²⁺, Li⁺, Na⁺ and HPO₄²⁻) and chaotropic ions have a low charge density (examples include H₂PO₄⁻, HSO₄⁻, HCO₃⁻, I⁻, Cl⁻, NO₃⁻, NH₄⁺, Cs⁺, K⁺, [N(CH₃)₄]⁺). The salting out salt can also be described in terms of its ability to donate or accept protons, and as such acting as a base or acid. For instance, the salting out salt (NH₄)₂SO₄ provides an ammonium ion, and can act as an inorganic acid. When included in a polymeric microparticle such inorganic acids can modulate polymer degradation and affect release of incorporated agent. In certain embodiments, amino acids such as glycine which is considered in the art as a kosmotrope can be used as an alternative to the salting-out salt.

Suitable salting-out salts for use in this invention include, for example, salts containing one or more of the cations Mg⁺², Li⁺, Na⁺, K⁺ and NH₄⁺; and also containing one or more of the anions SO₄⁻², HPO₄⁻², acetate, citrate, tartrate, Cl⁻, NO₃⁻, ClO₃⁻, I⁻, ClO₄⁻ and SCN⁻.

The amount of salting-out salt present in the sustained release composition can range from about 0.01% (w/w) to about 50% (w/w), such as from about 0.01% to about 10% (w/w), for example from about 0.01% to about 5%, such as 0.1% to about 5% of the total weight of the sustained release composition. Combinations of two or more salting-out salts can be used. The amount of salting-out salt, when a combination is employed, is the same as the range recited above.

### Sugars

A sugar, as defined herein, is a monosaccharide, disaccharide or oligosaccharide (from about 3 to about 10 monosaccharides) or a derivative thereof. For example, sugar alcohols of monosaccharides are suitable derivatives included in the present definition of sugar. As such, the sugar alcohol mannitol, for example, which is derived from the monosaccharide mannose is included in the definition of sugar as used herein.

Suitable monosaccharides include, but are not limited to, glucose, fructose and mannose. A disaccharide, as further defined herein, is a compound which upon hydrolysis yields two molecules of a monosaccharide. Suitable disaccharides include, but are not limited to, sucrose, lactose and trehalose. Suitable oligosaccharides include, but are not limited to, raffinose and acarbose.

The amount of sugar present in the sustained release composition can range from about 0.01% (w/w) to about 50% (w/w), such as from about 0.01% (w/w) to about 10% (w/w), such as from about 0.1% (w/w) to about 5% (w/w) of the total weight of the sustained release composition. Excellent release profiles were obtained incorporating about 2% (w/w) sucrose in a microparticle loaded with exendin-4.

Alternatively, the amount of sugar present in the sustained release composition can be referred to on a weight ratio with the agent or biologically active polypeptide. For example, the polypeptide and sugar can be present in a ratio from about 10:1 to about 1:10 weight:weight. In a particularly preferred embodiment, the ratio of polypeptide (e.g., exendin-4) to sugar (e.g., sucrose) is about 5:2 (w/w).

Combinations of two or more sugars can also be used. The amount of sugar, when a combination is employed, is the same as the ranges recited above.

When the polypeptide is exendin-4, the sugar is preferably sucrose, mannitol or a combination thereof.

### Surfactants

A surfactant can be present in the sustained release composition. The surfactant can act to further modify release of the biologically active polypeptide from the polymer matrix, or can act to further stabilize the biologically active polypeptide or a combination thereof. The presence of surfactant can in some instances assist in minimizing adsorption of the biologically active polypeptide to the biocompatible polymer. The amount of surfactant present in the sustained release composition can range from about 0.1% w/w to about 50% w/w of the dry weight of the composition.

Surfactant, as the term is used herein refers to any substance which can reduce the surface tension between immiscible liquids. Suitable surfactants which can be added to the sustained release composition include polymer surfactants, such as nonionic polymer surfactants, for example, poloxamers, polysorbates, polyethylene glycols (PEGs), polyoxyethylene fatty acid esters, polyvinylpyrrolidone and combinations thereof. Examples of poloxamers suitable for use in the invention include poloxamer 407 sold under the trademark PLURONIC® F127, and poloxamer 188 sold under the trademark PLURONIC® F68, both available from BASF Wyandotte. Examples of polysorbates suitable for use in the invention include polysorbate 20 sold under the trademark TWEEN® 20 and polysorbate 80 sold under the trademark TWEEN® 80.

Cationic surfactants, for example, benzalkonium chloride, are also suitable for use in the invention. In addition, bile salts, such as deoxycholate and glycocholate are suitable as surfactants based on their highly effective nature as detergents.

### Exendin-4 Compositions

A preferred embodiment of the present invention is a composition for sustained delivery of Exendin-4 made by the processes disclosed herein, and comprising a biocompatible polymer, the active agent, and a sugar.

### Manufacturing Processes

The present invention relates to methods of forming compositions for the sustained delivery of active agents. These methods are based on the coacervation process, which includes forming a first phase comprising the active agent, the polymer and a solvent; adding a coacervation agent, for example silicone oil, vegetable oil or mineral oil to the first phase to form embryonic microparticles; transferring the embryonic microparticles to a quench solvent to harden the microparticles; collecting the hardened microparticles; and drying the hardened microparticles. The first phase can be a water-in-oil emulsion prepared by combining an aqueous solution of the active agent with a solution of the polymer in an organic solvent. In this case, the process is generally referred to herein as a water-oil-oil process (W/O/O). Alternatively, the first phase can be a suspension of solid particles of the active agent in a solution of the polymer in an organic solvent. This alternative process is referred to herein as a solid-oil-oil process (S/O/O).

Preferably, the polymer can be present in the organic solvent at a concentration ranging from about 3% w/w to about 25% w/w, preferably, from about 4% w/w to about 15% w/w, such as from about 5% w/w to about 10% w/w. Where the polymer is a PLG, such as those preferred herein, the polymer is dissolved in a solvent for PLG. Such solvents are well known in the art, and are selected from the group consisting of alcohols, esters, ketones, halogenated hydrocarbons and blends thereof. Preferred solvents are methylene chloride (MeCl₂) and ethyl acetate.

The agent and water-soluble excipients, such as a sugar, are typically added in the aqueous phase, preferably in the same aqueous phase. The concentration of agent is preferably 10 to 100 mg/g, more preferably between 50 to 100 mg/g. The concentration of sugar is preferably 10 to 50 mg/g and more preferably 30 to 50 mg/g.

The solutions of the active agent and polymer are then mixed to form a water-in-oil emulsion, which is referred to herein as a first phase. It is preferred that the first phase emulsion be formed such that the inner emulsion droplet size is less than about 1 micron, preferably less than about 0.7 microns, more preferably less than about 0.5 microns, such as about 0.4 microns. Sonicators and homogenizers can be used to form such an emulsion.

In the embodiment of the coacervation process wherein the active agent is dispersed in the polymer solution as a solid (i.e., the S/O/O process), it is preferred that the suspended drug particle diameter be less than about 10 microns, preferably less than about 5 microns, and more preferably less than about 1 micron. Methods of producing submicron particles of biologically active agents are known in the art. For example, U.S. Pat. No. 6,428,815 discloses a spray-freeze drying process capable of producing friable microstructures which can be fragmented in the polymer solution to achieve submicron particles by means known to those skilled in the art, for example by probe sonication, homogenization, fluidization, comminution and milling.

### Coacervate Formation

A coacervation agent as used herein refers to a non-solvent for the polymer that is miscible with the polymer solvent. Coacervation agents may be low molecular weight polymer non-solvents. Alternatively, the coacervation agent may be a second polymer that is incompatible with the polymer that forms the microparticle. Addition of a coacervation agent reduces the solubility of the polymer, causing it to undergo phase separation, thus forming a coacervate. Suitable coacervation agents for use in the present invention include, but are not limited to, silicone oil, vegetable oil and mineral oil. In a particular embodiment, the coacervation agent is silicone oil and the polymer solvent is methylene chloride. Silicone oil is added in an amount sufficient to achieve a final silicone oil to methylene chloride ratio from about 0.75:1 to about 2:1. In a preferred embodiment, the final ratio of silicone oil to methylene chloride is from about 1:1 to about 1.5:1. In a more preferred embodiment, the final ratio of silicone oil to methylene chloride is about 1.3:1. In processes that employ coacervation agents other than silicone oil or polymer solvents other than methylene chloride, a suitable ratio of coacervation agent to polymer solvent can be selected on the basis of experimentally determined effects of the amount of coacervation agent on the microparticle size distribution, residual solvent and coacervation agent levels, and drug release kinetics.

The behavior of the nascent microparticles during formation of the coacervate is dependent on the process conditions. At low coacervation agent to polymer solvent ratios, i.e., after partial addition of the coacervation agent, the particle size of the nascent microparticles tends to be relatively stable. However, at higher ratios of coacervation agent to polymer solvent, i.e., near or prior to the completion of coacervation agent addition, nascent microparticles are prone to growth or coalescence, which can result in an unacceptably large particle size distribution in the final microparticle composition, or unacceptable yield losses if a finishing step is performed to remove oversized particles. With specific regard to the case where the coacervation agent is silicone oil, the polymer is PLG and the solvent is methylene chloride, at silicone oil to methylene chloride ratios of 0.5:1 or less, the particle size of the nascent microparticles is comparatively stable. At silicone oil to methylene chloride ratios of 0.7:1 or greater, the particle diameter increases over time. It is therefore preferred to limit the time interval during the coacervation step wherein the ratio of coacervation agent to polymer solvent is high enough to promote growth of the nascent particles. When the polymer solvent is methylene chloride and the coacervation agent is silicone oil, this time interval is preferably less than about 10 minutes and more preferably less than about 5 minutes.

The rate of addition of coacervation agent to the first phase, and the efficiency with which the coacervation agent and the first phase are blended, can also impact characteristics for the microparticle composition. In the case where the coacervation agent is silicone oil, the polymer is PLG and the solvent is methylene chloride, high mass flow rates of silicone oil can result in high residual silicone oil levels in the microparticles due to entrapment of silicone oil in the nascent microparticles. The level of residual silicone oil in the microparticles is most dependent on the addition rate at the early stages of silicone oil addition, for example when the ratio of silicone oil to methylene chloride is 0.5:1 or less. In order to control the level of residual silicone oil in the microparticles, silicone oil is preferably added slowly up to a ratio of silicone oil to methylene chloride of, for example, about 0.375:1. The rate of silicone oil addition is selected such that this phase of silicone oil addition occurs over a time interval of preferably greater than about 3 minutes, and more preferably over a period of at least about 5 minutes. In processes wherein the polymer solvent is not methylene chloride or the coacervation agent is not silicone oil, the minimum time for coacervation agent addition can be determined experimentally using methods known in the art. The early stages of coacervation agent addition to the first phase are preferably conducted under conditions where the coacervation agent is well dispersed in and efficiently blended with the first phase. Means of increasing the efficiency of blending include conducting the coacervation step in a stirred tank reactor outfitted with multiple addition ports or by adding the coacervation agent to the first phase through one or more spray nozzles.

The present invention includes coacervation processes wherein the addition of a coacervation agent to the first phase is conducted in a single stage, or in multiple stages, as disclosed in co-pending U.S. Patent Application No. 60/919,378. In processes comprising multiple stages of coacervation agent addition, the early stage or stages of coacervation agent addition typically takes place under conditions that avoid entrapment of coacervation agent in the microparticles, i.e., by slow addition of coacervation agent using equipment that promotes efficient blending of the coacervation agent with the first phase. The later stage or stages of the coacervation step typically take place under conditions designed to control particle size growth, such as minimizing the time interval during which the ratio of coacervation agent to polymer solvent is high enough to promote particle size growth.

The individual stages of coacervation agent may all take place in a single apparatus, or they may take place as separate unit operations in distinct pieces of equipment. Moreover, the individual stages of coacervation agent addition may all be conducted in either batch mode or continuous mode, or certain stages may be run as batch operations and others as continuous processes. Furthermore, it may be appropriate to introduce hold times between different stages of coacervation agent addition in order to allow time dependent processes to occur, such as, for example, diffusion of the polymer solvent out of the nascent microparticle. Alternatively different stages of coacervation agent addition may be made to take place in immediate succession, with no hold time in between.

Microparticle Quenching

The coacervate, formed either by a multistage process by a conventional single stage process, is combined with a quench liquid and the nascent microparticles are allowed to harden. In the present invention, the quench liquid is a solvent mixture comprising a hardening solvent and a washing solvent. A suitable hardening solvent is a non-solvent for the polymer but is miscible with the coacervation agent and the polymer solvent. Suitable hardening solvents include, but are not limited to, liquid hydrocarbons including heptane, hexane, pentane and cyclohexane; diethyl ether; petroleum ether; mineral oil; fatty acid esters; and caprylate triglyceride. Preferred hardening agents include liquid hydrocarbons. Of these, heptane is particularly preferred. Suitable washing solvents include, but are not limited to, alcohols, such as ethanol and isopropanol. In a preferred embodiment of the present invention, the hardening solvent is a liquid alkane, and the washing solvent is an alcohol. In a more preferred embodiment, the hardening solvent is heptane and the washing solvent is ethanol.

The makeup of the quench liquid may be selected on the basis of various considerations including flammability and static charge dissipation, cost and availability of raw materials, and the properties of the final microparticle product, including residual solvent levels, particle size, handing characteristics, yield and active agent release kinetics. In the process wherein the hardening solvent is heptane and the washing solvent is ethanol, there is an optimal range for quench liquid heptane-ethanol ratio. Heptane-ethanol mixtures containing less than 90% heptane have higher electrical conductivity and therefore dissipate static charge more efficiently than quench liquids described in the prior art that contain at least 90% heptane. In addition, for a fixed volume of quench liquid, lowering the heptane:ethanol ratio reduces the consumption of heptane, which is at times subject to worldwide shortages. Moreover, we have unexpectedly discovered that quench liquids containing less than 90% heptane enable the use of smaller volumes of quench liquid to harden the microparticles without appreciable negative effects on residual solvent levels, particle size or yield. At the same time, the amount of heptane in the quench liquid must be high enough to afford satisfactory microparticle handling characteristics. U.S. Pat. No. 6,824,822 discloses a process wherein the quench liquid is a 50:50 heptane-ethanol mixture. Microparticles produced using this process displayed unacceptable handling characteristics.

Accordingly, for processes wherein the quench liquid is a heptane-ethanol mixture, the heptane-ethanol ratio is preferably between about 90:10 and about 50:50 by weight. More preferably, the heptane-ethanol ratio is between about 80:20 and 65:35 by weight. Still more preferably, the heptane-ethanol ratio is about 75:25. For quench liquids comprising hardening solvents or washing solvents other than heptane and ethanol, a suitable quench liquid composition can be ascertained by one of skill in the art based on the similar considerations.

Microparticle quenching can be conducted in a batch mode in a stirred tank reactor. Typically this process is conducted at a controlled temperature, usually below ambient, such as about 3° C., with sufficient agitation to maintain the microparticles in a suspended state. In order to produce microparticles with acceptable residual solvent levels and handling characteristics, the volume of quench liquid must be sufficient to provide an adequate sink for the coacervation agent and the polymer solvent. Minimizing the amount of quench liquid required for a given microparticle batch size is also desirable from a process economics standpoint, since quench liquid volume is an important contributor to overall solvent consumption and capital equipment costs. Accordingly, it is preferred to optimize the quench liquid volume and composition in order to provide acceptable microparticles while minimizing solvent consumption and equipment size. For production of PLG microparticles in accordance with the process described n Example 1, i.e., wherein the polymer solvent is methylene chloride, the coacervation agent is silicone oil and nascent particles are quenched with 100% heptane or a heptane-ethanol mixture in which the ratio of heptane to ethanol is 90:10 or less, the necessary volume of quench liquid is at least about 16 times the volume of the polymer solvent. Use of a quench liquid containing heptane and ethanol at a ratio of 75:25, in accordance with the present invention, affords the opportunity to reduce the quench liquid volume to 12 times the polymer solvent volume, with no appreciable detrimental effects on microparticle yield, particle size or residual solvent levels.

Alternatively, the coacervate may be contacted with the quench liquid in a continuous manner using a static mixer. Use of a static mixer to combine the coacervate with the quench liquid improves the scaleability of the process and affords a potential reduction in the consumption of quench liquid.

### Microparticle Washing

Following the quench step, microparticles may be subsequently washed or rinsed with a hardening solvent, a washing solvent, or a solvent blend comprising a hardening solvent and a washing solvent. Preferred hardening solvents and washing solvents are disclosed above in regard to microparticle quench liquids. The hardening solvents and washing solvents employed for microparticle washing may be either the same or different from the solvents employed in the quench step. When the same hardening and washing solvents are employed in quench and wash steps, the ratio of hardening solvent to washing solvent may be the same in both steps, or it may be different.

An aspect of the present invention is a coacervation process wherein the coacervate is contacted with a quench liquid comprising a hardening solvent and a washing solvent and subsequently, the hardened microparticles are washed with either a washing solvent or a solvent blend comprising a hardening solvent and a washing solvent.

Hardened microparticles may be washed in the same vessel employed in the quench step. In a typical process, at the end of the quench step, the agitation is discontinued in order to allow the microparticles to settle. The quench liquid is then decanted, the microparticles are resuspended in the non-aqueous washing medium and agitation is resumed in order to extract additional solvent from the microparticles. Alternatively, the hardened microparticles may be separated from the quench liquid by filtration, backflushed from the filter into the quench tank and then washed. The wash step is typically conducted under temperature control at below ambient temperature. The particular conditions of temperature, duration, wash liquid composition and volume, and agitation required in order to afford microparticles of acceptable size, residual solvent content, drug release kinetics and handling properties can be readily determined by one of skill in the art.

The washed microparticles are collected by filtration and may be subsequently rinsed in order to ensure maximum line transfer. The collected microparticles are subsequently dried by means known to one of skill in the art. Typical drying methods include vacuum drying and lyophilization.

The invention will now be further and specifically described by the following examples.

### EXAMPLES

### Example 1

Microparticles were prepared using a 105 gram batch size coacervation process. The composition and volume of the quench liquid and the composition of the microparticle washing system were varied. The resulting microparticles were characterized with respect to yield, particle size, and residual solvents.

### A. Inner Water-In-Oil Emulsion Formation

A water-in-oil emulsion was created with the aid of a sonicator (Vibracell VCX 750 with a ½″ probe (part #A07109PRB; Sonics and Materials Inc., Newtown, Conn.). The water phase of the emulsion was prepared by dissolving 2.1 g sucrose in 63 g water. The oil phase of the emulsion was prepared by dissolving PLG polymer (97.7 g of purified 50:50 DL4A PLG (Alkermes)) in methylene chloride (1530 g or 6% w/v)). The inner emulsion was formed by adding the water phase to the oil phase while stirring at 1400 to 1600 rpm and sonicating at 100% amplitude over about a five minute period at 2-8° C. The sonication scheme was 2 minutes of sonication, 1 minute hold, and 2 more minutes of sonication. This results in an inner emulsion droplet size of less than 0.5 microns.

### B. Coacervate Formation

A coacervation step was then performed by adding silicone oil (2294 g of Dimethicone, NF, 350 cs) over about a three to five minute time period to the inner emulsion. This is equivalent to a ratio of 1.5:1, silicone oil to methylene chloride. The methylene chloride from the polymer solution partitions into the silicone oil and begins to precipitate the polymer around the water phase, leading to microencapsulation. The resulting coacervate was permitted to stand for a short period of time, for example, from about 1 minute to about 5 minutes prior to proceeding to the microsphere hardening step.

### C. Microsphere Hardening and Rinse

The coacervate was then transferred into a quench liquid comprising a mixture of heptane and ethanol in a 3° C. cooled, stirred tank (˜250 rpm). The weight of quench liquid was either 12 or 16 times the weight of methylene chloride in the oil phase of the inner emulsion. The quench liquid was a heptane-ethanol mixture. The ratio of heptane to ethanol was either 90:10 or 75:25 by weight. After being quenched for 1 hour at 3° C., the solvent mixture was decanted and the microparticles were washed with either fresh 100% heptane (13 kg) or heptane-ethanol (75:25 by weight; 13 kg) at 3° C. for 1 hour to remove additional silicone oil, ethanol and methylene chloride. 100% heptane was employed to wash and the microparticles that had been quenched with heptane-ethanol 90:10. Microparticles that had been quenched with heptane-ethanol 75:25 were washed and with additional heptane-ethanol 75:25.

### D. Microsphere Drying and Collection

At the end of the wash step, the microspheres were transferred and collected on a 20-25 micron screen which acted as a dead-end filter. A final rinse with heptane or heptane-ethanol 75:25 (6 kg at 4° C.) was performed to ensure maximum line transfer. The rinse liquid was the same makeup as the liquid used to wash the microparticles. The microspheres were then dried with a purge of nitrogen gas. The temperature was increased according to the following schedule: 18 hours at 3° C.; 24 hours at 25° C.; 6 hours at 41° C.; and 24 hours at 45° C.

After the completion of drying, the microspheres were collected, weighed to determine the unsieved yield, and then sieved through a 150 μm sieve, weighed to calculate the % sieve yield, and stored at −80° C.

### Results

Microparticle batches were produced in accordance with the above procedure using a 90:10 heptane-ethanol quench and a quench liquid to methylene chloride ratio of 16:1 (n=8), a 90:10 heptane-ethanol quench and a quench liquid to methylene chloride ratio of 12:1 (n=2), a 75:25 heptane-ethanol quench and a quench liquid to methylene chloride ratio of 16:1 (n=2) and a 75:25 heptane-ethanol quench and a quench liquid to methylene chloride ratio of 12:1 (n=2). Raw data for microparticle batches produced using the 75:25 heptane-ethanol quench are compiled in Table 1.

TABLE 1
Raw data for testing 75:25 heptane:ethanol quench, wash, rinse at 16:1
and 12:1 quench:MeCl₂ ratio

| | Product Attribute | | | |
|---|---|---|---|---|
| | # 37-01-128 | # 37-01-144 | # 37-01-157 | # 37-01-173 |
| | Quench:MeCl₂ ratio | | | |
| | 16:1 | | 12:1 | |
| Pre-sieved yield (g) | 89.8 | 90.2 | 86.1 | 91.5 |
| Sieved yield (g), <150 μm | 75.3 | 75.9 | 85.7 | 84.8 |
| Sieve yield (%) | 83.9 | 84.1 | 99.5 | 92.7 |
| Unsonicated DV50 (μm) | 49.4 | 47.3 | 48.8 | 48.1 |
| Unsonicated DV90 (μm) | 77.8 | 79.6 | 75.2 | 78.2 |
| Sonicated DV50 (μm) | 45.9 | 42.7 | 47.2 | 45.1 |
| Sonicated DV90 (μm) | 64.9 | 63.4 | 67.9 | 68.6 |
| Sonicated DV90-DV10 | 38.3 | 41.0 | 41.6 | 44.5 |
| (μm) | | | | |
| Agglomeration index | 1.28 | 1.34 | 1.17 | 1.20 |
| Residual ethanol (wt %) | 0.162 | 0.170 | 0.266 | 0.260 |
| Residual methylene chloride | 0.179 | 0.155 | 0.271 | 0.246 |
| (wt %) | | | | |
| Residual heptane (wt %) | 1.009 | 1.236 | 1.073 | 1.352 |
| Residual silicon (ppm) | 100 | 156 | 161 | 162 |

### Microparticle Yield

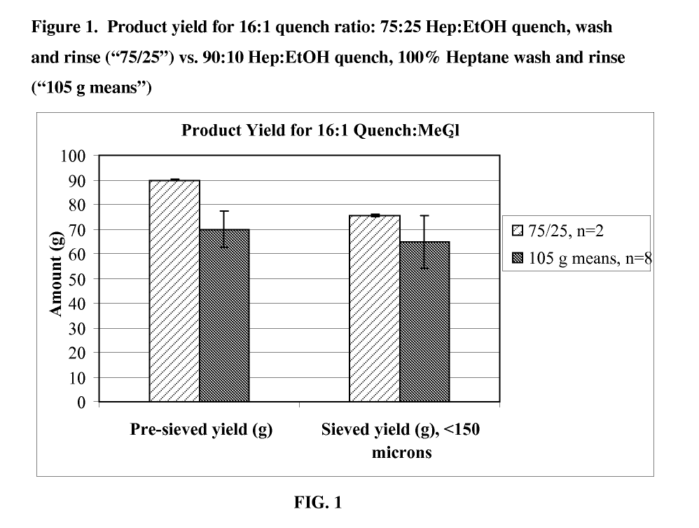

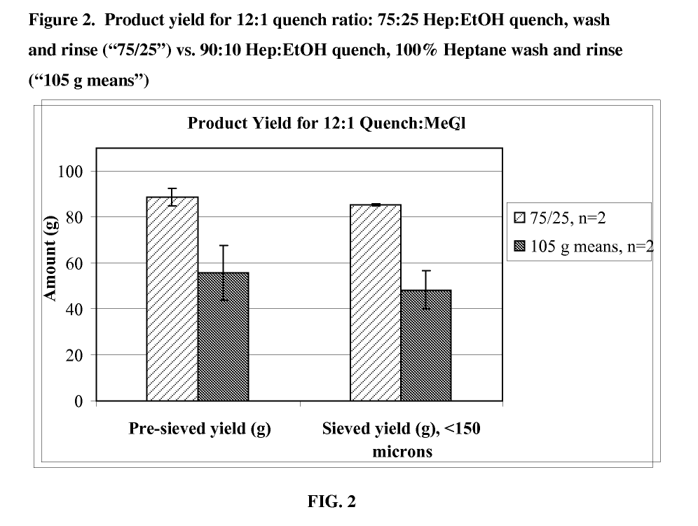

The weight of microparticles was determined gravimetrically both prior to and after sieving through a 150 μm sieve. Results are displayed in FIGS. 1 and 2. Higher yields were obtained when a 75:25 heptane-ethanol mixture was employed to quench, wash and rinse the microparticles. Reducing the weight of quench liquid to 12-times the weight of methylene chloride did not adversely affect product yield when the 75:25 heptane-ethanol mixture was used. On the other hand, very low microparticle yields were observed when the weight of heptane-ethanol 90:10 employed in the quench step was 12-times the weight of methylene chloride.

### Residual Solvents

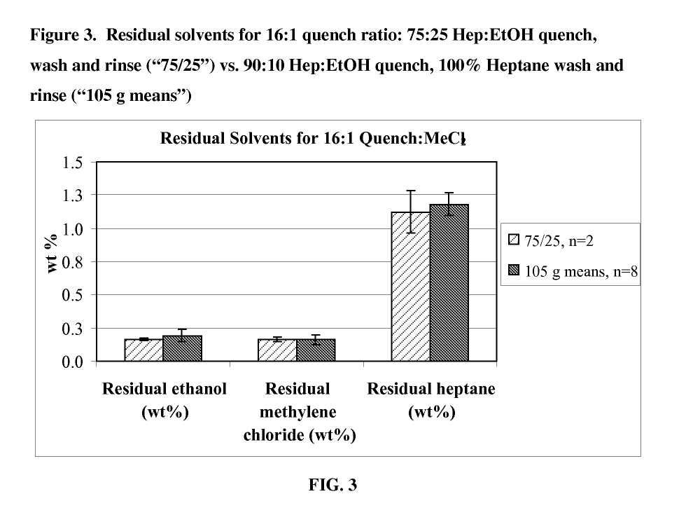

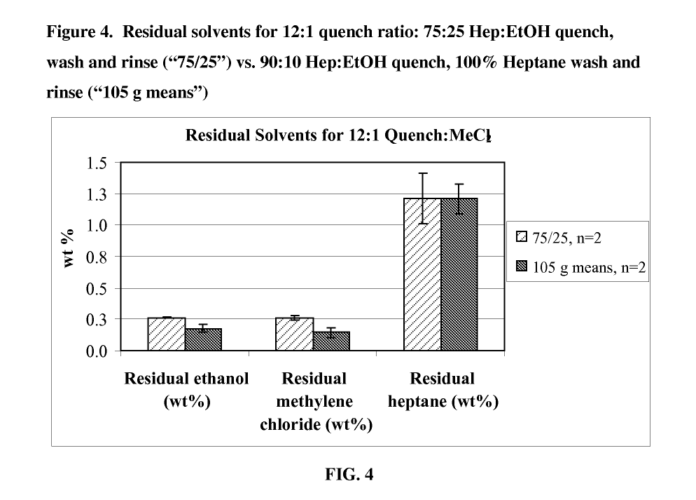

Residual solvent levels were determined using an HP 5890 Series 2 gas chromatograph with an Rtx 1301, 30 m×0.53 mm column. About 130 mg microparticles were dissolved in 10 ml N,N-dimethylformamide. Propyl acetate was used as the internal standard. The sample preparation was adjusted so that concentrations of methylene chloride as low as 0.03% can be quantitated. Residual solvent data are displayed in FIGS. 3 and 4.

### Particle Size

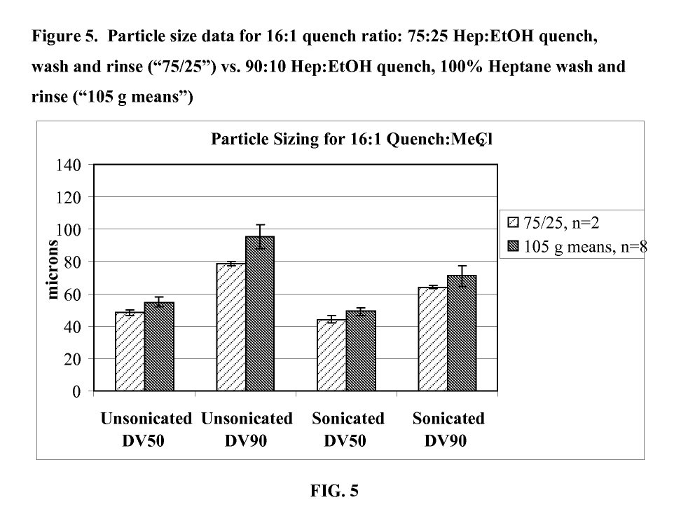

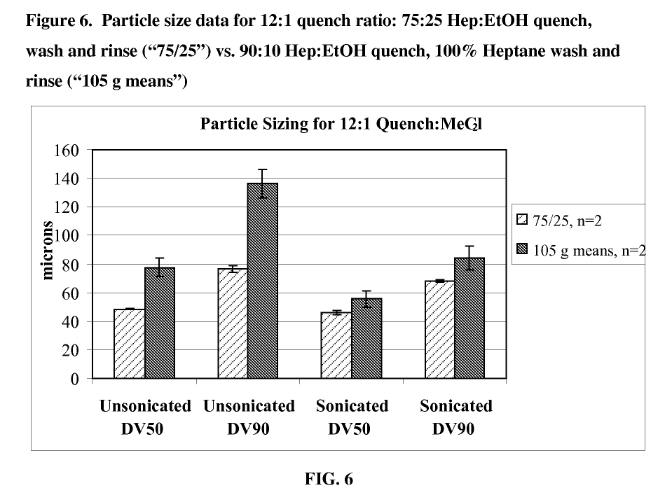

The particle size distribution of microparticles after suspension in an aqueous diluent containing 3% carboxymethylcellulose, 0.9% NaCl, 0.1% Tween 20 was obtained using a Coulter Multisizer. Particle size was determined with and without sonication of the microparticle suspension. DV50 is the mass median particle diameter; DV90 is the particle diameter at the 90^{th} volume percentile. Particle size data are displayed in FIGS. 5 and 6. The microparticles produced using the heptane-ethanol 75:25 quench liquid were of smaller overall size than those produced using the 90:10 heptane ethanol ratio in the quench.

The batches produced using the heptane-ethanol 75:25 quench were also characterized with respect to their agglomeration index (Table 1). The agglomeration index is the ratio of unsonicated DV95 to sonicated DV95. A lower agglomeration index is indicative of the ease of dispersibility of the particles in an aqueous diluent, which is desirable in a clinical setting. The microparticles produced using the heptane-ethanol 75:25 quench had agglomeration indices ranging from 1.17 to 1.34. These values compare favorably to an average (±std dev) of 1.44±0.15 μm for batches quenched with heptane-ethanol 90:10 at a quench ratio of 16:1, and 1.71±0.25 for batches quenched with heptane-ethanol 90:10 at a quench ratio of 12:1.

### Residual Silicon

Residual silicon was measured by inductively coupled plasma spectroscopy (ICP; Galbraith Laboratories, Inc; Knoxville, Tenn.). Residual silicon levels in batches made using a heptane-ethanol 75:25 quench (Table 1) are similar to the average value (±std dev) of 136±47 observed with batches quenched with heptane-ethanol 90:10.

### Example 2

Microparticles were prepared using a large scale coacervation process. A 20 kg batch was produced using a 75:25 heptane-ethanol mixture for the quench, wash and rinse steps, and a quench liquid to methylene chloride ratio of 12:1. For comparison, four 15 kg reference batches were produced using a 90:10 heptane-ethanol mixture for the quench step, 100% heptane for wash and rinse steps, and a quench liquid to methylene chloride ratio of 16:1. The resulting microparticles were characterized with respect to yield, particle size, and residual solvents.

### A. Inner Water-In-Oil Emulsion Formation

A water-in-oil emulsion was created with the aid of an in-line Megatron homogenizer MT-V 3-65 F/FF/FF, Kinematica AG, Switzerland. For the 20 kg batch, the water phase of the emulsion was prepared by dissolving 400 g sucrose in 12 kg water for irrigation (WFI). The oil phase of the emulsion was prepared by dissolving PLG polymer (e.g., 18,600 g of 50:50 DL4A PLG (Alkermes, Inc.)) in methylene chloride (292 kg or 6% w/w). For each 15 kg reference batch, 300 g sucrose was dissolved in 9 kg WFI, and 13950 g PLG was dissolved in 219 kg methylene chloride.

The water phase was added to the oil phase to form a coarse emulsion with an overhead mixer for about three minutes. Then, the coarse emulsion was homogenized at approximately 10,000 rpm at 5° C.

### B. Coacervate Formation

A coacervation step was then performed by adding silicone oil (Dimethicone, NF, 350 cs) to the inner emulsion in an amount sufficient to provide a silicone oil to methylene chloride ratio of about 1.5:1. The silicone oil addition profile was as follows: Silicone oil was added at a rate of 14 kg/min for 6 minutes (resulting in an intermediate silicone oil to methylene chloride ratio of 0.375:1). After a hold time of 5 minutes, additional silicone oil was added at 60 kg/min for 4 minutes.

### C. Microsphere Hardening and Rinse

The embryonic microspheres were then transferred into a 3° C. cooled, stirred tank containing 3500 kg quench liquid. In the 20 kg batch the quench liquid was a 75:25 w/w heptane-ethanol blend. In the reference batches, the heptane-ethanol ratio was 90:10 w/w. After being quenched for 1 hour at 3° C., the solvent mixture was decanted and then the microparticles were washed at 3° C. for 1 hour with 825 kg of 75:25 w/w heptane-ethanol (20 kg batch) or 100% heptane (15 kg reference batches).

### D. Microsphere Drying and Collection

At the end of the quench or decant/wash step, the microspheres were transferred and collected on a jacketed 0.2 m² filter dryer (3V Cogeim) equipped with a 20 micron Teflon membrane and a glycol-filled agitator for mixing and heat transfer during collection and drying. The microparticles were rinsed (four 150 kg rinses at 3° C.) with 75:25 w/w heptane-ethanol (20 kg batch) or 100% heptane (15 kg reference batches). The microparticles were then dried under vacuum with a constant purge of nitrogen gas at a controlled rate. The 20 kg batch was dried according to the following schedule: 6 hours at 3° C.; 36 hours ramping to 39° C.; and 64 hours at 39° C. A 72 hour in-process sample was collected after 30 hours of drying at 39° C. (72 hours total drying time). The same schedule was employed for the 15 kg reference batches except that the 39° C. drying step was carried out for 30 hours. Microparticles were sifted through a 150 μm screen and evaluated with respect to yield, particle size and residual solvents.

Particle size and yield data for the 20 kg batch are compared to the 15 kg reference batches in Table 2.

TABLE 2
Particle size and yield data for 20 kg batch and 15 kg reference batches

| | | Mean of 4 15 kg |
|---|---|---|
| | 20 kg batch | reference batches |
| Dv10 (μm) | 39 | 48 |
| Dv50 (μm) | 66 | 80 |
| Dv90 (μm) | 99 | 114 |
| Yield | 15.1 kg (out of | 12.0 (out of 14.3 kg) |
| | 19 kg) 79% | 84% |

Residual solvent data for the 20 kg batch and the in-process sample are compared to the 15 kg reference batches in Table 3.

TABLE 3
Residual solvents and silicon levels for 20 kg batch
and 15 kg reference batches

| | | 20 kg batch 72 hr | Mean of 4 15 kg |
|---|---|---|---|
| | 20 kg batch | in-process sample | reference batches |
| Residual EtOH | 0.26% | 0.44% | 0.58% |
| Residual MeCl₂ | 0.26% | 0.38% | 0.46% |
| Residual heptane | 0.92% | 0.93% | 0.73% |
| Residual silicon | <12 ppm | | 63 ppm |

Modifications and variations of the invention will be obvious to those skilled in the art from the foregoing detailed description of the invention. Such modifications and variations are intended to come within the scope of the appended claims.

All patents, patent application publications and articles cited herein are incorporated by reference in their entirety.

What is claimed:

1. A method for preparing microparticles comprising:
(a) providing a first phase comprising an active agent, a biocompatible polymer and a polymer solvent;
(b) forming a coacervate; and
(c) combining the coacervate with a quench liquid comprising a hardening solvent and between about 15% and about 45% by weight of a washing solvent, thereby forming microparticles containing the active agent.

2. The method of claim 1, wherein the average particle size of the microparticles is between about 1 um and about 250 um.

3. The method of claim 1, wherein the average particle size of the microparticles is between about 10 um and about 100 um.

4. The method of claim 1, wherein the active agent is a water soluble drug.

5. The method of claim 1, wherein the active agent is a biologically active polypeptide.

6. The method of claim 5, wherein the biologically active polypeptide is selected from glucagon, glucagon-like peptides, exendins, agonists of glucagon like peptides, vasoactive intestinal peptide, immunoglobulins, antibodies, cytokines, interleukins, macrophage activating factors, interferons, erythropoietin, tumor necrosis factor, colony stimulating factors, insulin, enzymes, tumor suppressors, blood proteins, follicle stimulating hormone, growth hormone, adrenocorticotropic hormone, and luteinizing hormone releasing hormone, NGF, EGF, gastrin, GRH, defensin, enkephalins, and muteins, analogs, deletion and substitution variants and pharmaceutically acceptable salts thereof.

7. The method of claim 5, wherein the biologically active polypeptide is a glucoregulatory peptide.

8. The method of claim 5, wherein the polypeptide is present from about 0.1% w/w to about 10% w/w of the total dry weight of the microparticles.

9. The method of claim 5, wherein the polypeptide is present from about 1% w/w to about 5% w/w of the total dry weight of the microparticles.

10. The method of claim 5, wherein the polypeptide is present from about 3% w/w to about 5% w/w of the total dry weight of the microparticles.

11. The method of claim 1, wherein the biocompatible polymer is selected from the group consisting of poly(lactides), poly(glycolides), poly(lactide-co-glycolides), poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polycyanoacrylates, poly(p-dioxanone), poly(alkylene oxalate)s, biodegradable polyurethanes, blends thereof and copolymers thereof.

12. The method of claim 11, wherein said polymer comprises poly(lactide-co-glycolide).

13. The method of claim 11, wherein said polymer is a 50:50 poly(lactide-co-glycolide).

14. The method of claim 13, wherein said polymer has an inherent viscosity of between about 0.3 and 0.5 dL/g.

15. The method of claim 11, wherein the polymer comprises a poly(lactide-co-glycolide) having a carboxylic acid end group.

16. The method of claim 1, wherein the polymer solvent is selected from the group consisting of alcohols, esters, ketones, halogenated hydrocarbons and blends thereof.

17. The method of claim 16, wherein the polymer solvent is methylene chloride.

18. The method of claim 16, wherein the polymer solvent is ethyl acetate.

19. The method of claim 1, wherein the first phase is a water-in-oil emulsion comprising the active agent dissolved in the aqueous dispersed phase of the emulsion.

20. The method of claim 19, wherein the water-in-oil emulsion has a dispersed phase droplet size of less than 1 um.

21. The method of claim 20, wherein the water-in-oil emulsion has a dispersed phase droplet size of 0.2 um to 0.4 um.

22. The method of claim 19, wherein the concentration of active agent in the aqueous phase of the water-in-oil emulsion is 10 mg/g to 100 mg/g.

23. The method of claim 22, wherein the concentration of active agent in the aqueous phase of the water-in-oil emulsion is 50 mg/g to 100 mg/g.

24. The method of claim 1, wherein the first phase is a suspension of particles comprising the active agent in a solution comprising the polymer and the polymer solvent.

25. The method of claim 24, wherein the average diameter of the suspended particles is less than about 10 um.

26. The method of claim 25, wherein the average diameter of the suspended particles is less than about 1 um.

27. The method of claim 1, wherein the first phase comprises the active agent dissolved in the polymer solvent.

28. The method of claim 1, wherein the first phase further comprises an excipient selected from the group consisting of salts, sugars, carbohydrates, buffers and surfactants.

29. The method of claim 28, wherein the excipient is a sugar.

30. The method of claim 29, wherein the sugar is selected from a monosaccharide, a disaccharide, a sugar alcohol or a combination thereof.

31. The method of claim 30, wherein the sugar is selected from sucrose, mannitol and combinations thereof.

32. The method of claim 31, wherein the sugar is sucrose.

33. The method of claim 29, wherein the amount of sugar in the microparticles is 0.01% to 10% of the total dry weight of the microparticles.

34. The method of claim 28, wherein the excipient and the active agent are dispersed together in the first phase.

35. The method of claim 33, wherein the amount of sugar in the microparticles is 1% to 5% of the total dry weight of the microparticles.

36. The method of claim 33, wherein the amount of sugar in the microparticles is about 2% of the total dry weight of the microparticles.

37. The method of claim 28, wherein the total amount of drug and excipient in the microparticles is less than 10% of the total dry weight of the microparticles.

38. The method of claim 37, wherein the total amount of drug and excipient in the microparticles is between 3% and 8% of the total dry weight of the microparticles.

39. The method of claim 38, wherein the total amount of drug and excipient in the microparticles is between 5% and 7% of the total dry weight of the microparticles.

40. The method of claim 1, wherein the ratio of polymer to polymer solvent in the first phase is about 20% or less.

41. The method of claim 34, wherein the ratio of polymer to polymer solvent in the first phase is about 10% or less.

42. The method of claim 1, wherein the coacervation agent comprises an oil.

43. The method of claim 42, wherein the oil is selected from the group consisting of mineral oil, vegetable oil and silicone oil.

44. The method of claim 43, wherein the oil is silicone oil.

45. The method of claim 1, wherein the weight ratio of coacervation agent to polymer solvent in step (b) is from about 0.75:1 to about 2:1.

46. The method of claim 45, wherein the weight ratio of coacervation agent to polymer solvent in step (b) is from about 1.0:1 to about 1.5:1.

47. The method of claim 1, wherein the hardening solvent comprises a hydrocarbon.

48. The method of claim 47, wherein the hydrocarbon is heptane.

49. The method of claim 1, wherein the washing solvent comprises an alcohol.

50. The method of claim 49, wherein the alcohol is ethanol.

51. The method of claim 48, wherein the quench liquid comprises between about 20% and about 35% of a washing solvent.

52. The method of claim 49, wherein the quench liquid comprises about 25% of a washing solvent.

53. The method of claim 1, wherein the quench liquid comprises a mixture of heptane and ethanol at a ratio from about 55:45 to about 85:15.

54. The method of claim 53, wherein the quench liquid comprises a mixture of heptane and ethanol at a ratio from about 65:35 to about 80:20.

55. The method of claim 53, wherein the quench liquid comprises a mixture of heptane and ethanol at a ratio of about 75:25.

56. The method of claim 1, wherein the ratio of quench liquid to polymer solvent is less than about 15:1 by weight.

57. The method of claim 56, wherein the ratio of quench liquid to polymer solvent is about 12:1 by weight.

58. The method of claim 1 further comprising:
(d) washing the microparticles with a liquid comprising a hardening solvent.

59. The method of claim 58, wherein the hardening solvent in step (d) is the same as the hardening solvent in step (c).

60. The method of claim 59, wherein the hardening solvent is heptane.

61. The method of claim 58, wherein the liquid in step (d) further comprises a washing solvent.

62. The method of claim 58, wherein the washing solvent and hardening solvent in step (d) are the same as the washing solvent and hardening solvent in step (c).

63. The method of claim 62, wherein the washing solvent is ethanol and the hardening solvent is heptane.

64. The method of claim 63, wherein the hardening solvent to washing solvent is in a ratio of about 85:15 by weight to about 55:45 by weight.

65. The method of claim 1 further comprising:
(d) collecting the microparticles, and
(e) drying the microparticles.

66. The method of claim 58 further comprising:
(e) collecting the microparticles; and
(f) drying the microparticles.

67. A method for preparing microparticles comprising:
(a) providing a first phase comprising an active agent, a biocompatible polymer and a polymer solvent;
(b) forming a coacervate;
(c) combining the coacervate with a quench liquid comprising a hardening solvent and a washing solvent, thereby forming microparticles containing the active agent; and
(d) washing the microparticles with a washing solvent or a solvent mixture comprising a hardening solvent and a washing solvent.

68. The method of claim 67, wherein the hardening solvent and washing solvent of step (c) are the same as the hardening solvent and washing solvent of step (d).

69. The method of claim 68, wherein the quench liquid of step (c) and the solvent mixture of step (d) comprise heptane and ethanol.

70. The method of claim 69, wherein the quench liquid of step (c) and the solvent mixture of step (d) comprise heptane and ethanol in a ratio from about 95:5 to about 50:50.

71. The method of claim 70, wherein the quench liquid of step (c) and the solvent mixture of step (d) comprise heptane and ethanol in a ratio from about 90:10 to about 60:40.

72. The method of claim 71, wherein the quench liquid of step (c) and the solvent mixture of step (d) comprise heptane and ethanol in a ratio from about 80:20 to about 70:30.

73. The method of claim 72, wherein the quench liquid of step (c) and the solvent mixture of step (d) comprise heptane and ethanol in a ratio of about 75:25.

74. Microparticles prepared by the method of claim 1.

75. Microparticles prepared by the method of claim 67.
